SEQUENCE LISTING (44 sequences)

SEQ ID NO: 1 - protein, 5 aa, synthetic construct
DRGMG

SEQ ID NO: 2 - protein, 5 aa, synthetic construct
NHGMG

SEQ ID NO: 3 - protein, 5 aa, synthetic construct
RHGMG

SEQ ID NO: 4 - protein, 20 aa, synthetic construct
GVDDSGSSRDYAPAVKGRAT

SEQ ID NO: 5 - protein, 21 aa, synthetic construct
GINSGAGSYTNYGAAVKGRAT

SEQ ID NO: 6 - protein, 20 aa, synthetic construct
SINSGGSYTNYGAAVQGRAT

SEQ ID NO: 7 - protein, 9 aa, synthetic construct
GSVVGFIDA

SEQ ID NO: 8 - protein, 16 aa, synthetic construct
SVYGGSISLITDVIDT

SEQ ID NO: 9 - protein, 19 aa, synthetic construct
DAGCTGGGTCYAYADRIDA

SEQ ID NO: 10 - protein, 11 aa, synthetic construct
SGGGSYAGSYY

SEQ ID NO: 11 - protein, 13 aa, synthetic construct
SGGANYYGSYYYG

SEQ ID NO: 12 - protein, 13 aa, synthetic construct
SGGGRFAGSYYYG

SEQ ID NO: 13 - protein, 8 aa, synthetic construct
SGSSGSYG

SEQ ID NO: 14 - protein, 9 aa, synthetic construct
SGSYSNYYG

SEQ ID NO: 15 - protein, 6 aa, synthetic construct
NNNNRP

SEQ ID NO: 16 - protein, 6 aa, synthetic construct
DNTKRP

SEQ ID NO: 17 - protein, 6 aa, synthetic construct
DSTNRP

SEQ ID NO: 18 - protein, 6 aa, synthetic construct
YNGKRP

SEQ ID NO: 19 - protein, 6 aa, synthetic construct
ENNNRP

SEQ ID NO: 20 - protein, 10 aa, synthetic construct
GSIDSSYVGI

SEQ ID NO: 21 - protein, 11 aa, synthetic construct
GSYDSSAGYAG

SEQ ID NO: 22 - protein, 10 aa, synthetic construct
GSRDSSYDGI

SEQ ID NO: 23 - protein, 11 aa, synthetic construct
GGYDDSTDSGI

SEQ ID NO: 24 - protein, 243 aa, synthetic construct
AVTLDESGGGLQTPGGALSLVCKASGFTFSDRGMGWMRQAPGKGLEFVAGVDDSGSSRDYAPAVKGRATISRDNGQSTLSLQLSDLRAEDTGTYYCTRGSVVGFIDAWGHGTEVIVSSGGGGSGGGGSGGGGSQAALTQPASVSANPGETVKITCSGGGSYAGSYYYGWYQQKSPGSAPVTVIYNNNNRPSGIPSRFSGSLSGSTNTLTITGVQAEDEAIYYCGSIDSSYVGIFGAGTALTVL

SEQ ID NO: 25 - protein, 253 aa, synthetic construct
AVTLDESGGGLQTPGGGLSLVCKASGFTFSNHGMGWVRQAPGKGLEWVAGINSGAGSYTNYGAAVKGRATISRDNGQSTVRLQLNNLRAEDTGTYFCARSVYGGSISLITDVIDTWGHGTEVTVSSGGGGSGGGGSGGGGSQAALTQPASVSANLGETVKITCSGGANYYGSYYYGWYQQKSPGSAPVTVINDNTKRPSNIPSRFSGSTSGSTGTLTITGVQADDEAVYFCGSYDSSAGYAGIFGAGTTLTVL

SEQ ID NO: 26 - protein, 243 aa, synthetic construct
AVTLDESGGGLQTPGGTVSLVCKASGFTFSDRGMGWMRQAPGKGLEFVAGVDDSGSSRDYAPAVKGRATISRDNGQDILRLQLNNLRAEDTATYYCTRGSVVGFIDAWGHGTEVIVSSGGGGSGGGGSGGGGSQAALTQPASVSASPGETVKITCSGGGRFAGSYYYGWYQQKSPGSAPVTVIYDSTNRPSNIPSRFSGSLSGSTTTLTITGVQADDEAVYFCGSIDSSYVGIFGAGTTLTVL

SEQ ID NO: 27 - protein, 248 aa, synthetic construct
AVTLDESGGGLQTPGGALSLVCKASGFSFSRHGMGWVRQAPGKGLEWVASINSGGSYTNYGAAVQGRATIWRDNGQSTVRLQLNNLRAEDTATYYCARDAGCTGGGTCYAYADRIDAWGHGTEVIVSSGGGGSGGGGSGGGGSQAALTLPSSVSVNPGETVKITCSGSSGSYGWYQQKSPGSAPVTVIYYNGKRPSDIPSRFSGSKSGSTGTLTITGVQAEDEAVYFCGSRDSSYDGIFGAGTTLTVL

SEQ ID NO: 28 - protein, 248 aa, synthetic construct; identical to SEQ ID NO: 27

SEQ ID NO: 29 - protein, 31 aa, synthetic construct
GQPAGPDNKENCPVPGKPGEAVAARKLFAPQ

SEQ ID NO: 30 - protein, 729 aa, synthetic construct
GCTGTAACGCTTGATGAATCTGGTGGGGGTTTACAAACACCGGGAGGAGCATTGTCGCTCGTTTGTAAGGCCAGCGGGTTCACATTCAGCGACAGGGGTATGGGATGGATGAGGCAAGCGCCCGGAAAAGGGCTAGAGTTTGTTGCGGGTGTAGATGATTCGGGTAGCAGTAGAGATTATGCCCCCGCAGTGAAAGGTAGGGCTACAATATCTAGGGATAACGGCCAGAGCACGCTAAGCCTCCAATTAAGTGATCTTCGCGCTGAAGACACGGGCACGTATTATTGCACACGGGGTTCCGTAGTAGGGTTCATAGACGCATGGGGTCACGGGACGGAAGTCATAGTATCATCAGGGGGAGGCGGATCCGGTGGCGGGGGATCCGGAGGTGGTGGTTCCCAGGCAGCGTTGACACAGCCGGCGTCAGTCAGTGCGAACCCAGGGGAAACGGTGAAAATTACGTGCTCCGGTGGAGGCTCTTATGCAGGGTCTTATTACTATGGTTGGTATCAACAAAAATCTCCTGGCAGTGCCCCAGTCACTGTCATTTATAATAACAACAACCGACCTAGTGGTATTCCGTCGCGTTTTAGCGGTTCCCTTTCGGGCTCCACGAATACCCTCACGATTACGGGCGTCCAAGCTGAAGACGAAGCGATCTACTACTGCGGATCTATCGACTCTTCCTACGTAGGTATATTTGGTGCCGGCACCGCTTTGACTGTATTA

SEQ ID NO: 31 - protein, 34 aa, synthetic construct
DRGMGGVDDSGSSRDYAPAVKGRATGSVVGFIDA

SEQ ID NO: 32 - protein, 27 aa, synthetic construct
SGGGSYAGSYYNNNNRPGSIDSSYVGI

SEQ ID NO: 33 - protein, 759 aa, synthetic construct
GCGGTAACATTAGACGAGTCAGGCGGGGGACTTCAAACGCCGGGAGGGGGCTTGTCTCTAGTATGCAAAGCGTCTGGATTTACCTTCAGCAATCATGGCATGGGGTGGGTGCGCCAAGCTCCTGGAAAAGGCTTGGAGTGGGTTGCAGGAATCAACAGCGGGGCAGGCTCTTACACAAACTACGGTGCAGCAGTAAAAGGTAGGGCAACCATCAGCCGAGACAACGGACAAAGCACAGTCAGGTTGCAGCTCAACAATTTAAGGGCAGAAGACACTGGCACATACTTCTGCGCACGGTCTGTCTACGGCGGTAGCATATCTCTCATAACGGATGTAATCGACACGTGGGGCCATGGGACGGAAGTGACAGTTTCATCCGGTGGTGGAGGGAGTGGTGGTGGAGGCTCCGGTGGTGGAGGGTCACAAGCAGCCCTGACACAACCGGCAAGCGTCTCAGCTAACTTGGGAGAAACAGTTAAAATCACCTGCTCTGGTGGTGCTAATTACTATGGCAGCTATTATTACGGATGGTACCAACAAAAAAGTCCAGGGTCAGCACCTGTCACAGTCATCAATGACAACACGAAACGCCCATCTAACATACCGTCCAGGTTTTCTGGCAGCACAAGCGGAAGTACAGGCACTCTCACAATTACGGGAGTGCAGGCCGACGATGAAGCTGTCTATTTCTGTGGCTCTTACGACTCTAGCGCTGGCTACGCAGGTATATTTGGAGCAGGTACAACTTTAACAGTATTA

SEQ ID NO: 34 - protein, 42 aa, synthetic construct
NHGMGGINSGAGSYTNYGAAVKGRATSVYGGSISLITDVIDT

SEQ ID NO: 35 - protein, 30 aa, synthetic construct
SGGANYYGSYYYGDNTKRPGSYDSSAGYAG

SEQ ID NO: 36 - protein, 729 aa, synthetic construct
GCAGTAACATTGGATGAATCCGGTGGGGGTTTGCAAACTCCTGGTGGGACAGTCTCACTCGTGTGTAAGGCGTCAGGTTTCACTTTTTCCGATCGGGGTATGGGATGGATGCGGCAAGCGCCGGGCAAAGGACTTGAATTCGTAGCCGGAGTAGATGACTCTGGGTCCAGTCGTGACTACGCGCCCGCTGTCAAAGGTCGGGCGACGATAAGTCGTGACAATGGTCAAGACATATTACGGCTCCAGCTAAACAATCTACGTGCTGAAGATACAGCAACATATTATTGTACGAGAGGCTCAGTGGTAGGTTTCATAGATGCATGGGGACACGGCACGGAGGTTATAGTTTCCTCCGGTGGGGGAGGCTCTGGAGGCGGCGGTTCTGGCGGCGGAGGGTCACAAGCGGCGTTAACCCAACCCGCGTCAGTCTCAGCTTCCCCCGGGGAAACGGTCAAGATCACCTGTTCCGGGGGGGGGCGTTTTGCTGGATCTTATTACTATGGATGGTATCAACAGAAATCCCCCGGTAGTGCCCCGGTGACCGTTATTTACGACTCGACAAATCGGCCTTCTAATATACCGAGTCGTTTCTCTGGTAGCTTGTCTGGCTCAACAACAACGCTAACGATAACCGGTGTTCAGGCAGATGACGAGGCTGTGTATTTCTGCGGCTCTATCGACAGCAGCTATGTAGGGATATTCGGAGCTGGCACGACTCTTACCGTGTTA

SEQ ID NO: 37 - protein, 34 aa, synthetic construct; identical to SEQ ID NO: 31

SEQ ID NO: 38 - protein, 29 aa, synthetic construct
SGGGRFAGSYYYGDSTNRPGSIDSSYVGI

SEQ ID NO: 39 - protein, 744 aa, synthetic construct
GCTGTCACTCTAGACGAGTCAGGAGGCGGACTCCAGACACCTGGGGGGGCCCTAAGCTTGGTTTGTAAAGCATCAGGGTTTTCCTTTTCACGACATGGAATGGGATGGGTCCGGCAAGCCCCGGGAAAAGGACTGGAGTGGGTTGCGAGTATTAATTCAGGGGGATCGTATACGAACTATGGCGCAGCCGTGCAGGGAAGAGCGACTATATGGCGTGATAATGGTCAATCAACGGTAAGGCTACAATTGAATAACCTCCGGGCCGAGGATACGGCAACCTATTATTGTGCGCGAGATGCGGGATGCACTGGTGGCGGCACATGTTATGCATACGCGGACCGTATTGATGCGTGGGGACATGGGACGGAAGTTATTGTATCTAGTGGGGGCGGAGGCTCTGGGGGGGGGGGGTCGGGCGGTGGGGGCTCCCAGGCAGCGTTAACGCTCCCGAGCTCTGTGTCCGTGAATCCAGGAGAAACTGTTAAAATCACATGTTCAGGCTCTAGCGGCAGCTACGGCTGGTATCAACAAAAATCCCCCGGTTCCGCTCCAGTCACCGTTATTTACTACAATGGGAAGAGGCCGTCGGATATACCGTCTCGCTTCTCCGGTTCTAAATCCGGCTCAACGGGGACACTCACCATAACTGGCGTACAAGCTGAGGACGAGGCGGTGTACTTTTGCGGTAGTCGTGATAGCTCATACGACGGCATATTTGGAGCTGGTACAACACTGACGGTGCTT

SEQ ID NO: 40 - protein, 44 aa, synthetic construct
RHGMGSINSGGSYTNYGAAVQGRATDAGCTGGGTCYAYADRIDA

SEQ ID NO: 41 - protein, 24 aa, synthetic construct
SGSSGSYGYNGKRPGSRDSSYDGI

SEQ ID NO: 42 - protein, 744 aa, synthetic construct
GCGGTTACACTAGATGAGAGCGGAGGTGGTCTACAAACGCCTGGTGGGGGTTTATCCCTCGTATGTAAAGCGTCTGGCTTCACATTTTCCAACCACGGGATGGGATGGGTACGCCAGGCACCCGGTAAAGGTCTCGAATGGGTGGCTGGAATAAATAGCGGAGCAGGATCTTATACCAACTACGGCGCAGCCGTTAAGGGGCGTGCAACTATCTCCCGCGACAATGGACAGTCCACCGTTCGACTCCAACTGAATAACCTTCGGGCTGAGGACACTGCTACTTATTACTGCGCCCGTTCGGTTTATGGTGGGAGTATTTCGTTAATTACAGATGTTATTGACACATGGGGCCACGGCACTGAGGTCATCGTCTCTTCTGGTGGGGGCGGCTCAGGAGGAGGCGGGTCTGGAGGAGGCGGTTCGCAGGCGGCCCTTACGCAACCATCAAGTGTCAGTGCAAATCCGGGAGAAACTGTTAAGATAACCTGTTCCGGATCTTACAGCAATTATTATGGCTGGTATCAGCAAAAATCTCCGGGGAGTGCTCCTGTTACTGTGATATACGAAAACAACAACAGGCCCAGCGATATTCCTTCACGTTTTTCGGGATCTAAATCTGGTTCTACTAACACTTTGACAATTACTGGTGTCCAGGTAGAGGACGAAGCCGTGTACTTTTGCGGCGGTTACGATGACTCTACGGACAGCGGAATATTCGGCGCAGGCACCACGCTGACCGTACTT

SEQ ID NO: 43 - protein, 42 aa, synthetic construct; identical to SEQ ID NO: 34

SEQ ID NO: 44 - protein, 26 aa, synthetic construct
SGSYSNYYGENNNRPGGYDDSTDSGI

(19) United States
(12) Patent Application Publication
(10) Pub. No.: US 2025/0244331 A1
(43) Pub. Date: Jul. 31, 2025

(54) CHICKEN-DERIVED RECOMBINANT FULL-LENGTH MONOCLONAL IGY ANTIBODY AGAINST HUMAN THYMIDINE KINASE 1 AND APPLICATIONS THEREOF

(71) Applicant: SINO-SWED TONGKANG BIO-TECH (SHENZHEN) LIMITED, Shenzhen (CN)

(72) Inventors: Ailian HEI, Shenzhen (CN); Jin LI, Shenzhen (CN); Ellen HE, Shenzhen (CN); Sven Isac SKOG, Shenzhen (CN); Ji ZHOU, Shenzhen (CN)

(73) Assignee: SINO-SWED TONGKANG BIO-TECH (SHENZHEN) LIMITED, Shenzhen (CN)

(21) Appl. No.: 19/173,763

(22) Filed: Apr. 8, 2025

Related U.S. Application Data
(63) Continuation of application No. PCT/CN2023/102914, filed on Jun. 27, 2023.

(30) Foreign Application Priority Data
Oct. 10, 2022 (CN) 202211233518.0

(51) Int. Cl. G01N 33/574; C07K 16/40; G01N 33/573

(52) U.S. Cl. CPC G01N 33/57484; C07K 16/40; C12Y 207/01021; G01N 33/573; C07K 2317/24; G01N 2333/9122

(57) ABSTRACT

Disclosed are a chicken-derived recombinant full-length monoclonal IgY antibody against human TK1 and applications thereof. Through an improved phage display method, a human TK1 C-terminal 195-225 polypeptide was used to develop a chicken-derived recombinant full-length monoclonal IgY antibody against human TK1. A 3.4×10⁹ pfu/mL ultra-large library was quickly constructed within three weeks, with a positive cloning rate of up to 98%. ELISA, immunoblotting and immunohistochemical detection methods were used to identify a preferred hTK1-IgY-rmAb antibody with high specificity and high sensitivity. The invention improves the stability of antibody batches, realizes the operation of a new generation of TK1 detection kit based on recombinant IgY antibodies on a fully automatic chemiluminometer, and can meet the detection needs of large-scale early tumor risk screening.

### CROSS-REFERENCE TO RELATED APPLICATIONS

The present application is a continuation application of International Application No. PCT/CN2023/102914, filed on Jun. 27, 2023, which claims priority to Chinese Patent Application No. 202211233518.0, filed on Oct. 10, 2022. The entire contents of the above-mentioned applications are incorporated herein by reference.

### REFERENCE TO SEQUENCE LISTING SUBMITTED ELECTRONICALLY

A Sequence Listing is provided herewith as a Sequence Listing XML, “SEQUENCE LISTING.xml” created on Feb. 28, 2025 and having a size of 44,483 bytes. The contents of the Sequence Listing XML are incorporated by reference herein in their entirety.

### TECHNICAL FIELD

The present application relates to the technical field of antibody engineering, and in particular to a chicken-derived recombinant full-length monoclonal IgY antibody against human TK1 and applications thereof.

### BACKGROUND

Thymidine kinase 1 (TK1) is a kinase in the pyrimidine salvage pathway. With the participation of magnesium ions and ATP, TK1 catalyzes the conversion of deoxythymidine (dT) into deoxythymidine monophosphate (dTMP), participates in the synthesis of DNA in the S phase of the cell cycle, and is a key enzyme in cell cycle regulation. TK1 is a key enzyme involved in DNA synthesis during the cell cycle and is closely related to the rate of cell proliferation. The content of TK1 in the serum of normal healthy individuals without any tumor disease is extremely low (<2.0 pmol/L), but under the abnormal proliferation of tumor cells, TK1 can be easily detected in the serum and tissues of tumor patients. The increasing of TK1 concentration in serum indicates the signal of abnormal cell proliferation. The increase of TK1 is significantly correlated with the proliferation rate of tumor cells in patients especially with malignant tumors. It is a recognized that TK1 is an accurate biomarker for evaluating the proliferation of tumor cells, used for early risk screening, evaluation of therapeutic efficacy for surgery, radiotherapy and chemotherapy, and prediction of tumor recurrence risk in various types of malignant tumors.

Currently, there are a series of mouse IgG monoclonal antibodies (mAbs) against human TK1 on the market, such as those from AroCell (www.arocell.com) in Sweden, and rabbit-derived anti-human TK1 recombinant monoclonal antibodies, such as those from Abcam (https://www.abcam.com) in the United States. However, regarding these antibodies for serological TK1 detection, most are only used for scientific research, lacking serological large-scale clinical detection performance verification. Although these mAbs can be used to evaluate the treatment effects of cancer patients through serological TK1 levels, due to their insufficient specificity and sensitivity, they cannot be used for early detection of tumors and risk progression assessment in health checkup screenings, nor are they suitable for the prognosis evaluation of tumor patients. No large-sample literature data has been published yet.

The commercial enhanced immunodot chemiluminescence method (ECL-dot blot) developed by SINO-SWED TONGKANG BIO-TECH (SHENZHEN) LIMITED uses the C-terminal peptide (195-225) of human TK1 to develop chicken anti-human TK1-IgY polyclonal antibody (TK1-IgY-pAbs), which has been successfully used to detect the concentration of thymidine kinase 1 protein (STK1p) in human serum. It has been applied in clinical tumors and physical examination screening for more than 15 years, verifying that TK1-IgY-pAbs is not only a reliable and precise target for evaluating the outcome and prognosis of tumor patients, but can also be used to evaluate the prognosis, recurrence risk and survival effect of tumor patients. In particular, it can be used for risk warning assessment of early tumor progression. Based on the data from 160,086 individuals undergoing routine health checkups at four independent centers in China from 2005 to 2016, high STK1p values detected by TK1-IgY-pAbs can indicate the risk level of benign and malignant tumors, i.e. low-risk or high-risk groups, before the occurrence of imaging abnormalities. This is suitable to various occupations and groups, and is particularly valuable for early tumor risk screening and health management in cancer-prone regions, such as industries involving exposure to carcinogenic chemicals, achieving the goal of “early screening, early prevention, early detection, and early treatment of tumors”.

For the previous generation of products, due to the individual differences and living environment of the immunized hens, it is necessary to regularly test whether each hen produces antibodies with high stability, good specificity and high sensitivity in order to control the detection performance of each production batch of antibodies. These control tasks not only require sophisticated preparation processes and experienced technicians, but also take a long time and have high costs. Therefore, the IgY polyclonal antibody products obtained based on the previous generation of traditional immunized hens are in urgent need of further improvement.

### SUMMARY

In order to improve the stability of antibody batches, the present application provides a chicken-derived recombinant full-length monoclonal IgY antibody against human TK1 and their applications.

In a first aspect, the present application initially provides a chicken-derived recombinant IgY single-chain antibody against human TK1, the single-chain antibody is composed of a heavy chain, a linker peptide and a light chain, the heavy chain includes a first heavy chain variable region, a second heavy chain variable region and a third heavy chain variable region, and the light chain of the single-chain antibody includes a first light chain variable region, a second light chain variable region and a third light chain variable region. The amino acid sequence of the first heavy chain variable region is selected from one of SEQ ID NO: 1 to SEQ ID NO: 3, the amino acid sequence of the second heavy chain variable region is selected from one of SEQ ID NO: 4 to SEQ ID NO: 6, and the amino acid sequence of the third heavy chain variable region is selected from one of SEQ ID NO: 7 to SEQ ID NO: 9. The amino acid sequence of the first light chain variable region is selected from one of SEQ ID NO: 10 to SEQ ID NO: 14, the amino acid sequence of the second light chain variable region is selected from one of SEQ ID NO: 15 to SEQ ID NO: 19, and the amino acid sequence of the third light chain variable region is selected from one of SEQ ID NO: 20 to SEQ ID NO: 23.

In some embodiments, the variable region of the same single-chain antibody consists of SEQ ID NO: 1, SEQ ID NO: 4, SEQ ID NO: 7 of the heavy chain and SEQ ID NO: 10, SEQ ID NO: 15, SEQ ID NO: 20 of the light chain, consists of SEQ ID NO: 2, SEQ ID NO: 5, SEQ ID NO: 8 of the heavy chain and SEQ ID NO: 11, SEQ ID NO: 16, SEQ ID NO: 21 of the light chain, consists of SEQ ID NO: 1, SEQ ID NO: 4, SEQ ID NO: 7 of the heavy chain and SEQ ID NO: 12, SEQ ID NO: 17, SEQ ID NO: 20 of the light chain, consists of SEQ ID NO: 3, SEQ ID NO: 6, SEQ ID NO: 9 of the heavy chain and SEQ ID NO: 13, SEQ ID NO: 18, SEQ ID NO: 22 of the light chain, or consists of SEQ ID NO: 2, SEQ ID NO: 5, SEQ ID NO: 8 of the heavy chain and SEQ ID NO: 14, SEQ ID NO: 19, SEQ ID NO: 23 of the light chain.

In a second aspect, the present application provides a chicken-derived recombinant full-length monoclonal IgY antibody against human TK1, including the chicken-derived recombinant IgY single-chain antibody against human TK1 described in the first aspect. In some embodiments, the chicken-derived recombinant full-length monoclonal IgY antibody against human TK1 has an amino acid sequence selected from one of SEQ ID NO: 24 to SEQ ID NO: 28.

In some embodiments, the amino acid sequence of the monoclonal antibody is the amino acid sequence of SEQ ID NO: 28.

In a third aspect, the present application provides a recombinant vector, including a nucleic acid encoding the amino acid sequence of the chicken-derived recombinant IgY single-chain antibody against human TK1 described in the first aspect or the amino acid sequence of the chicken-derived recombinant full-length monoclonal IgY antibody against human TK1 described in the second aspect, and the vector for constructing the recombinant vector is a PTT5 plasmid.

In a fourth aspect, the present application provides a cell containing a recombinant vector, the cell contains the recombinant vector described in the third aspect, and a stable cell line is constructed using Chinese hamster ovary (CHO) cells.

In a fifth aspect, the present application provides a test card, a test strip or a test kit, the test card, the test strip or the test kit contains any one of the chicken-derived recombinant IgY single-chain antibody against human TK1 described in the first aspect or the chicken-derived recombinant full-length monoclonal IgY antibody against human TK1 described in the second aspect.

In a sixth aspect, the present application provides a pharmaceutical composition, containing any one of the chicken-derived recombinant IgY single-chain antibody against human TK1 described in the first aspect or the chicken-derived recombinant full-length monoclonal IgY antibody against human TK1 described in the second aspect.

In a seventh aspect, the present application provides a method for screening the chicken-derived recombinant IgY single-chain antibody against human TK1 described in the first aspect or the chicken-derived recombinant full-length monoclonal IgY antibody against human TK 1 described in the second aspect, including the following steps:
step 1), isolating the mRMA of the chicken-derived recombinant IgY single-chain antibody against human TK1 described in the first aspect or the chicken-derived recombinant full-length monoclonal IgY antibody against human TK1 described in the second aspect from the hen spleen cells; step 2), performing polymerase chain reaction (PCR) reverse transcription; step 3), performing PCR overlap to obtain the full-length scFv gene; step 4), splicing and concentrating scFv; step 5), electrotransformation in a highly active bacterial competent state; step 6), phage antibody packaging; and step 7), screening. Using this method, a large-capacity phage antibody display library can be quickly constructed within three weeks.

In some embodiments, the step 1) includes rapidly taking spleen cells within 15 minutes after the hen is killed, homogenizing with TriZol solution, and isolating total RNA with chloroform and isopropanol.
The step 2) includes using oligodT as a primer to perform a reverse transcription reaction to prepare cDNA for subsequent amplification of antibody genes.
The step 3) includes using degenerate primers to amplify the genes of the heavy chain variable region and the light chain variable region, and assembling the genes into scFv genes by overlapping PCR.
The step 4) includes isolating the PCR product and cutting the PCR product with Sfi I and Not I, and ligating the PCR product into the phagemid vector pSCD-1 (as shown in FIG. 1) using T4 DNA ligase.
The step 5) includes desalting the ligation mixture and transforming the ligation mixture via electroporation into TG1 competent cells.
The step 6) includes adding the helper phage M13KO7 to the phagemid vector shown in FIG. 1, and precipitating and titrating the phage obtained by amplification after packaging is ed by PEG 6000 for further screening.
The step 7) includes adding the phage library to a screening plate containing the target TK1, digesting and eluting the bound phage after multiple washing and incubation, amplifying and screening for multiple rounds, taking out a single colony for inoculation and culture, adding auxiliary phage at a multiplicity of infection (MOI) of 10-20 for culturing when the absorbance value at a wavelength of 600 nm reaches 0.4-0.6, and performing enzyme-linked immunosorbent assay (ELISA) detection and DNA sequencing on the phage; according to results of the DNA sequencing, recombining all unique single-chain antibodies, based on the main chain of the heavy chain and light chain sequence of the IgY full-length antibody, constructing the full-length antibody sequence through continuous PCR and cloning steps, and then loading it into the eukaryotic expression vector PTT5 to obtain the PTT5-TK1-IgY-H and PTT5-TK1-IgY-L expression vector pairs.

In an eighth aspect, the present application provides a detection method, including any one of the chicken-derived recombinant IgY single-chain antibody against human TK1 described in the first aspect or the chicken-derived recombinant full-length monoclonal IgY antibody against human TK1 described in the second aspect, and manually or automatically detecting the TK1 concentration in a body fluid sample by enhanced chemiluminescence (ECL) dot blot or chemiluminescence immunoassay. In some embodiments, the body fluid is one selected from blood, urine, saliva, cerebrospinal fluid, and effusion.

In the previous generation of products, the inventors mastered how to selectively obtain TK1-IgY polyclonal antibodies with strong specificity and high sensitivity from immune hens. Drawing on the existing improved recombinant antibody development technology, its preparation process is easier to standardize, while reducing the consumption of time, manpower and cost costs, which is particularly important and necessary for the development of TK1-IgY monoclonal.

Based on the existing TK1-IgY polyclonal antibody technology, the inventors developed recombinant chicken IgY-TK1-mAb. Since recombinant monoclonal IgY antibodies are antibodies derived from a single B cell lineage, the lineage has undergone repeated somatic hypermutation and clonal selection, and is usually an IgY isotype that recognizes a single unique epitope. Phage display technology can now be used to successfully and quickly develop recombinant anti-TK1 monoclonal IgY antibodies. Compared with the corresponding natural polyclonal IgY antibodies, recombinant anti-TK1 monoclonal IgY antibodies have stronger specificity, higher affinity and stability. Recombinant antibodies also have the advantages of editability, and can be modified according to actual needs in terms of antibody stability, affinity, antibody modification, etc. to meet different new antibody application requirements. The inventor team successfully developed for the first time a chicken-derived recombinant full-length monoclonal IgY antibody against human TK1, named “human TK1 recombinant-IgY-monoclonal antibody”. The new generation of human TK1 recombinant-IgY-monoclonal antibody will provide innovative prospects for the research and application of early tumor risk screening and tumor medicine.

Cancer is a chronic tumor cell proliferation disease, mainly due to uncontrolled and unlimited cell proliferation caused by gene mutations in certain enzymes and proteins related to cell growth regulation, leading to the occurrence of malignant tumors. The non-invasive serological TK1 detection method provides information on the early risk process of tumor detection that cannot be achieved by imaging. Therefore, the serological TK1 biomarker is of great value in predicting the risk of malignant tumor progression. The serological TK1 marker combined with imaging and other applicable tools will enable long-term follow-up to detect precancerous lesions or early risk signs of tumors that cannot be detected by imaging. The inventors designed a specific TK1 C terminal-31 peptide antigen, successfully prepared chicken anti-human TK1-IgY antibodies, and developed and established a commercially available highly sensitive immunoenhanced chemiluminescent dot blot (ECL dot blot) detection system to measure the concentration of TK1 protein (STK1p) in serum, thereby replacing the detection method of TK1 activity (STKa) in serum. Based on a meta-study of TK1 in serum samples from a large dataset of 35,365 individuals undergoing health checkups, the inventors set STK1p=2.0 pM as a scientifically reasonable “risk threshold” to effectively evaluate the growth rate of abnormal cells in routine population screening. In this meta-study, the internationally recognized receiver operating characteristic (ROC) analysis method was used to analyze preoperative patients with 10 different types of malignant tumors (n=720) and individuals without malignant tumors/no tumor-related diseases (n=4,103), and the area under the curve was 0.96. When the optimal specificity was 0.997, the sensitivity was 0.798, and the maximum likelihood value (+) was 233.73, the risk threshold was 2.0 pM. The risk threshold of 2.0 pM was used to verify the effectiveness of the inventor's enhanced chemiluminescent dot blot immune system assembly TK1 kit in routine population physical examination screening applications. In this meta-study, it was found that the high-risk group of STK1p (STK1p>2.0 pM) had a 3-5 times higher incidence of new malignant tumors within 6 years than the new incidence rate of Chinese tumor statistics (0.2%-0.3%). Compared with the low-risk group of STK1p (≤2.0 pM), the high-risk group of STK1p had an even higher risk of new malignant tumors within 11 years (44 times). The inventor's research has confirmed that STK1p is more suitable for monitoring the efficacy of various types of tumors, assessing the recurrence and survival rate of cancer patients, and is particularly suitable for early detection of malignant tumors and predicting the risk process of tumor development. It is an optimal biomarker for predicting malignant tumors and predicting the risk of tumor development.

The present application uses the C-terminal 195-225 polypeptide of human TK1 to develop a chicken anti-human TK1 full-length IgY monoclonal antibody. Through the improved phage display method, the positive cloning rate is as high as 98%, and a 3.4×10⁹ pfu/mL ultra-large library is quickly constructed within 3 weeks, which can completely eliminate environmental phage contamination. After successfully obtaining a single-chain antibody that meets the inventor's requirements, the antibody heavy and light chain variable region sequences are obtained, and the obtained antibody fragments are analyzed to construct a full-length IgY antibody. Then, the full-length antibody is expressed through a eukaryotic expression system and purified. The antibody was tested by ELISA titer determination, immunoblotting and immunohistochemistry, which verified that the antibody was an antibody with high specificity and sensitivity. More importantly, serological identification pointed out that the results from two serum test kits assembled by the inventors showed a high concordance rate of r=0.857 between the two kits. In the future, the fully automatic chemiluminometer will replace the previous semi-automatic enhanced chemiluminescence immunoassay system, and the new generation of TK1-IgY recombinant antibody kits will be run on the fully automatic chemiluminometer, which has met the detection needs of large-scale early tumor risk screening. At present, the successful construction of TK1-IgY-rmAb antibody using phage display library technology and its operation on the fully automatic chemiluminometer has not been reported yet, which is an international first.

The inventors successfully obtained TK1-IgY-rmAb antibodies using phage display library technology, and now constructed a stable cell line, with the ultimate goal of preparing a large number of TK1-IgY-rmAb antibodies with high specificity, high sensitivity and stability, and assembling a detection kit for serum detection for early tumor risk screening in routine population physical examinations. In the future, the TK1-IgY-rmAb kit will show significant potential in early tumor risk screening for routine population health checkups. It can be used to predict precancerous diseases and/or tumor-related risk diseases, as well as to assess the risk prognosis of future progression to malignant tumors, and provide patients with timely early intervention and treatment. Furthermore, this new generation of TK1-IgY-rmAb kits are also suitable for clinical tumors, including follow-up and treatment effect monitoring of tumor patients, and prediction and evaluation of recurrence and survival.

Phage display technology has become an important method for preparing monoclonal antibodies from immune and non-immune sources. This method is simple, fast, and time-saving. It can quickly screen and obtain monoclonal antibodies without being limited by conventional hybridoma methods. It is currently the most mature and widely used genetic engineering antibody screening technology. Phage display technology has the advantages of large screening capacity, high efficiency, easy operation at the genetic level, and easy large-scale production. Its library capacity is as high as 10⁹-10¹² pfu/mL, which can select millions to hundreds of millions of molecules, and each round of panning can enrich clones with strong antigen specificity; making the screening of specific antibodies easier. This method can modify antibodies according to different purposes, broadening the scope of application of antibodies. Phage display technology has made great achievements in the application of antibodies, but most of the research is to obtain antibody libraries from immunized mammals. Studies have shown that some highly conserved mammalian antigens usually have low immunogenicity during the immunization of mammals.

### BRIEF DESCRIPTION OF THE DRAWINGS

FIG. 1 is a schematic diagram of phagemid pSCD-1.

FIG. 2A-2E illustrate the construction of phage antibody library, DNA (2000, 1000, 750, 500, 250 and 100 bp), where FIG. 2A is isolated RNA, FIG. 2B is the second round PCR of light chain of antibody, FIG. 2C is the second round PCR of heavy chain of antibody, FIG. 2D is overlapping PCR splicing scFv gene, and FIG. 2E is library diversity detection.

FIG. 3 is the eukaryotic expression of recombinant full-length protein TK1-IgY-rmAb 5^{#}, with antibody titer detection using Elisa titer assay (antibody concentration gradient dilution 1000-7.8 ng/mL). The upper right corner of FIG. 3 is the denaturing SDS-PAGE gel immunoelectrophoresis.

FIG. 4A shows TK1 immunohistochemistry (HIC) staining images in normal tonsil tissue and poorly differentiated ovarian cancer patient tissue.

FIG. 4B shows the results of TK1-IgY-rmAb 5^{#} for ECL dot blot assay and fully automated chemiluminescence assay.

FIG. 5 shows the results of hTK1-IgY-rmAb and hTK1-IgY-pAb for ECL dot blot assay.

FIG. 6 is a schematic diagram showing the antibody phage display library for screening of HTK1 scFv gene and reformatted and production of full-length recombinant HTK1-IgY mAb.

### DETAILED DESCRIPTION OF THE EMBODIMENTS

### EXAMPLE 1

This example discloses the preparation process of chicken-derived recombinant full-length monoclonal IgY antibody against human TK1.

### 1. Construction of TK1-IgY-scFv Phage Library

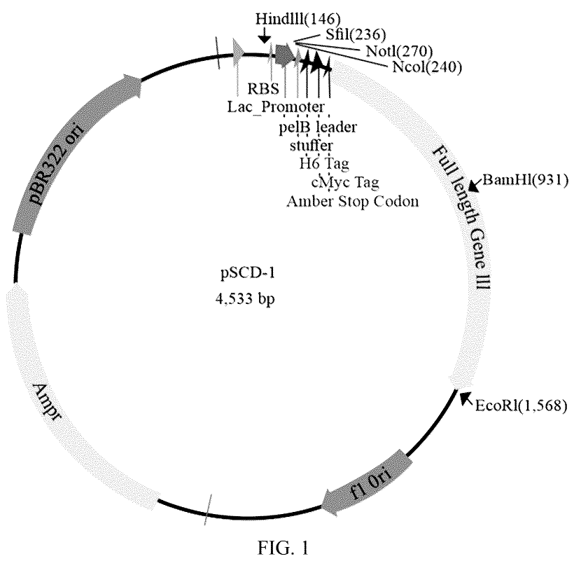

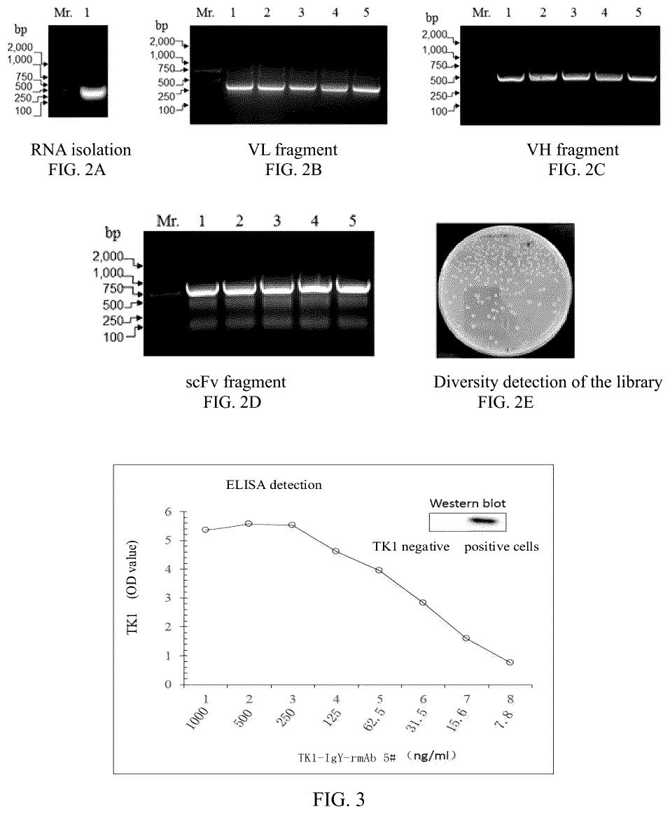

Step 1, isolating mRMA from hen spleen cells: immunize hens with a polymer of a synthetic human TK1-31 peptide (SEQ ID NO: 29) and keyhole limpet hemocyanin (KLH). Collect eggs and isolate crude TK1-IgY-pAb polyclonal crude from egg yolks, then obtain TK1-IgY-pAb purified antibodies by affinity purification, and identify the specificity and sensitivity of the antibodies by immunoblotting, immunohistochemistry and serology. The hens that produce TK1-IgY-Abs with high specificity and sensitivity are selected for library construction. Kill the hens, quickly take spleen cells within 15 minutes, and homogenize them with TriZol solution (Invitrogen).
Step 2, PCR reverse transcription, total RNA is isolated with chloroform and isopropanol, and then oligo dT is used as a primer for reverse transcription reaction to prepare cDNA. It is used for subsequent amplification of antibody genes.
Step 3, Overlapping polymerase chain reaction (PCR) to obtain full-length scFv gene: cDNA is prepared using the 1ₛₜ Strand cDNA Synthesis Kit from TaKaRa™ Prime Script. Degenerate primers are used to amplify VH and VL genes. They are assembled into scFv genes by overlapping PCR.
Step 4, Splicing concentrated scFv: PCR products are separated and cut with Sfi I and Not I before being ligated to the phagemid vector pSCD-1 (FIG. 1) by T4 DNA ligase.
Step 5, electrotransformation in highly active bacterial competent cells: desalting the ligation mixture and transforming the ligation mixture via electroporation into TG1 competent cells.
Step 6, phage antibody library packaging: adding the helper phage M13KO7 to the phagemid vector, and precipitating the packaged phage by PEG 6000 and routinely titrating to prepare for further screening. The structure of the library is shown in FIG. 2A-2E. The rapid construction of a large-capacity phage antibody display library was completed in 3 weeks, as shown herein. Finally, a phage library with antibody diversity was obtained, with a library size of 3.4×10⁹ pfu/ml (pfu, plaque forming unit) for subsequent screening. The calculation of the antibody library capacity is shown in Table 1. 50 single clones were randomly selected for colony PCR identification, and 49 clones had the correct amplification band size, with a positive clone rate of up to 98%.

TABLE 1
Storage capacity calculation method

Detection capacity plate number

| | 1 | total |
|---|---|---|
| Number of competent cells | 5 | 5 |
| Number of clones | 340 | 340 |

storage space = 5 × 200 × 10\hat{ }3 × (340/0.1) = 3.4 × 10⁹

### 2. Screening of Qualified Positive Clones

The phage library was added to a blocked 96-well plate coated with target TK1 and incubated at 37 is shown in Table 1. 50 single clones were randomly selected for colony by trypsin digestion. Finally, with the help of M13KO7 helper phage, the eluate was amplified and titrated for the next round of screening, and a total of three rounds of screening were performed. The content of Tween 20 in PBST was increased in each round (i.e., 0.1% Tween 20-PBS in the first round, 0.2% Tween 20-PBS in the second round, and 0.3% Tween 20-PBS in the third round), and the concentration of coated TK1 antigen was reduced from the initial concentration of 10 μg/mL to 2 μg/mL in each round.

The enrichment efficiency of each round was calculated. The calculation results showed that the ratio of output and input phages increased steadily after each round of panning (Table 2). Compared with the first round of screening, the phage recovery rate after the third round of screening increased by about 923 times, demonstrating the effective enrichment of specific antibodies. The panned library was used for the subsequent selection of high-affinity antibodies.

After the third round of panning, a single colony was picked from the final eluate (without amplification) and inoculated into a 96-well plate containing 100 g 100 nd of panning (Table 2). Compared with the first round of screening, the phage re° C./220 rpm. The next day, 20 μL of the overnight culture was transferred to a new 96-well plate containing 80 μL 2YT-A (2YT medium, 100 μg/mL ampicillin). When the OD 600 reached about 0.5, M13KO7 was added at an MOI of 10-20 and cultured at 37° C./180 rpm for 30 min. Subsequently, 100 μL 2YT-AK (2YT medium, 100 μg/mL ampicillin, 50 μg/mL kanamycin) was added to each well and cultured overnight at 37° C./220 rpm. Finally, 100 μL of supernatant per well was transferred to the assay plate coated with TK1 target for Phage Elisa detection.

TABLE 2
Phage library panning process.

| Number of panning | input (pfu) | output (pfu) | Enrichment rate |
|---|---|---|---|
| 1 | 5.0 × 10¹² | 9.6 × 10³ | 1.92 × 10⁻⁹ |
| 2 | 5.24 × 10¹² | 7.44 × 10⁴ | 1.42 × 10⁻⁸ |
| 3 | 5.28 × 10¹² | 9.36 × 10⁶ | 1.77 × 10⁻⁶ |

To identify the quality of the antibody library, sequencing of the antibody library is required: the positive clones selected by Phage ELISA were inoculated into LB-A (LB medium, 100 μg/mL ampicillin) and cultured overnight at 37° C./220 rpm. The plasmid of each clone was isolated using a commercial kit, and the sequence dat was analyzed using Geneious software after DNA sequencing.

A total of 49 samples were sequenced, with 44 successful sequencing results and 5 failure. 35 of the 44 clones were correct clones, and 9 had frameshift mutations. All 35 clones were different and were unique sequences.

The affinity was tested by limiting dilution method (TK1 antigen 0.125 μg-10 μg/well). The results showed that 5 positive clones had high affinity. The protein gene sequences corresponding to these 5 clones, the sequences of the variable regions include:
SEQ ID NO: 30, where the sequence of the heavy chain variable region was SEQ ID NO: 31, and the sequence of the light chain variable region was SEQ ID NO: 32;
SEQ ID NO: 33, where the sequence of the heavy chain variable region was SEQ ID NO: 34, and the sequence of the light chain variable region was SEQ ID NO: 35
SEQ ID NO: 36, where the sequence of the heavy chain variable region is SEQ ID NO: 37, and the sequence of the light chain variable region is SEQ ID NO: 38;
SEQ ID NO: 39, where the sequence of the heavy chain variable region is SEQ ID NO: 40, and the sequence of the light chain variable region is SEQ ID NO: 41;
SEQ ID NO: 42, where the sequence of the heavy chain variable region is SEQ ID NO: 43, and the sequence of the light chain variable region is SEQ ID NO: 44.

### 3. Eukaryotic Expression of Recombinant Full-Length Antibody Protein

The unique sequences of the above five scFv positive clones were screened and obtained. Five full-length IgY antibodies, SEQ ID NO: 24-28, were constructed, which were named TK1-hen-1, 2, 3, 4 and 5 respectively. First, they were transfected into 293T suspension cells for expression, and the supernatant after 6 days of culture was collected for Elisa detection.

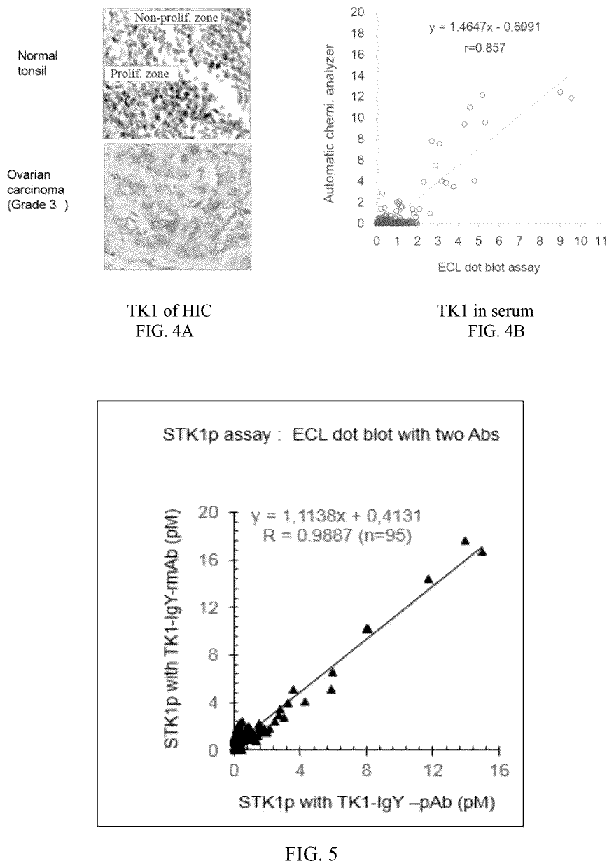

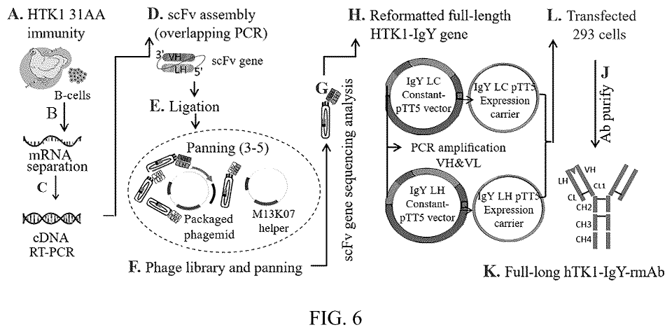

1 μg/mL TK1 target coated antigen was used for detection; the 5^{#} antibody with high affinity binding was selected from the five antibodies and named human TK1-IgY-rmAb 5^{#}. The sensitivity and specificity of the antibody were subsequently verified by immunoblotting, immunohistochemistry and serological tests (FIG. 3 and FIG. 4A-4B).

FIG. 3 is the ELISA titer method to detect TK1-IgY-rmAb 5^{#}: 1 μg/mL TK1 target was used as the coating antigen to detect the titer of the purified antibody, and the purified antibody was diluted to 7.8 ng/ml by 2-fold gradient dilution. Incubate at 37° C. for 1 h, then wash three times with PBST (0.1% Tween, PBS), then add the secondary antibody with HRP, wash five times with PBST, finally add TMB for color development, and detect at OD 450 after IM H2SO4 termination. Repeated detection results show (FIG. 3) that the antibody has high sensitivity.

TK1 negative cell line (143BTK1−/−) and TK1 positive cell line (HT29) were used for denaturing SDS-PAGE gel immunoelectrophoresis (Western blot) detection. 50 ng of cell lysate was loaded, and the amount of TK1-IgY-rmAb 5# used was 0.05 μg/mL. The test results showed that there was an obvious TK1 monomer (about 25 kD) in the TK1-positive cell lysate, which was a band specifically expressed by TK1. However, no TK1 monomer-specific band appeared in the TK1-negative cell lysate, proving that the antibody had good specificity.

### 4. TK1 Tissue Immunochemistry and TK1 Serological Identification of TK1-IgY-rmAb 5^{#}

Immunohistochemistry (TK1) staining was performed using a patient with poorly differentiated ovarian cancer (pathological grade 3) and normal tonsil tissue. Normal tonsil tissue contains 2 different areas, including proliferative and non-proliferative areas. It is often used as a good standard test method to evaluate whether an antibody can evaluate cell proliferation rate. Therefore, we used this tissue to evaluate the specificity and sensitivity of TK1-IgY-rmAb 5^{#}. The results in FIG. 4A show that TK1 staining was only observed in cells in the proliferative area of the tonsil. The staining was characterized by varying degrees of brown-yellow staining mainly in the cytoplasm, but no staining was observed in the cytoplasm of tonsil cells in the non-proliferative area. In addition, TK1 staining in patients with poorly differentiated ovarian cancer (pathological grade 3) is characterized by varying degrees of brown-yellow staining mainly in the cytoplasm. This demonstrates that TK1-IgY-rmAb 5^{#} has high specificity and sensitivity, which is the same as the IgY-pAb and TK1-IgG monoclonal antibodies we identified in the past.

The concentration of STK1p in 292 serum samples was detected using ECL dot blot and fully automatic chemiluminescence analyzer, and then compared and analyzed. The analysis results showed (FIG. 4B) that the concordance rate of the two methods was as high as r=0.857 (exceeding 0.75), indicating good agreement between the two detection methods, and the fully automatic chemiluminescence analyzer will be more conducive to large-scale early tumor risk screening.

### EXAMPLE 2

This example provides a method for preparing a recombinant vector containing a chicken-derived recombinant full-length monoclonal IgY antibody against human TK1, including the following steps:
Step 1), designing and synthesizing IgY heavy chain and light chain gene fragments: integrating the DNA fragment sequences corresponding to the heavy chain variable region (SEQ ID NO: 34) and light chain variable region (SEQ ID NO: 44) with the IgY backbone region DNA fragment sequence and adding an EcoR I restriction site sequence and a protective base to the front end of the DNA fragment, and adding a BamH I restriction site sequence and a protective base to the end; then performing full gene synthesis.
Step 2), loading the DNA fragment into the eukaryotic expression vector PTT5: double-digesting the synthesized DNA fragment with EcoR I and BamH I, followed by inserting between EcoR I/BamH I of the eukaryotic expression vector PTT5 to obtain a recombinant vector containing a recombinant chicken anti-human TK1 (5^{#}) full-length IgY monoclonal antibody.

### EXAMPLE 3

This example provides a cell preparation method for a recombinant vector containing a chicken-derived recombinant full-length monoclonal IgY antibody against human TK1 gene, including the following steps:
Step 1), adjusting the cell density: counting the 293T suspension cells, centrifuging and discarding the supernatant, adjusting the cell density to 4.0×10⁶ cells/mL, and culturing at 37° C. with a volume concentration of 5% CO₂ for 4 hours.
Step 2), transfection: adding the mixture to the transfection medium at a ratio of DNA (5^{#}) (μg): PEI (1 mg/mL)=1:4, vortexing to mix, and incubating at room temperature for 15 min; adding the mixture dropwise to the cell suspension to be transfected, and gently shaking while adding.
Step 3), cell culture: after transfection, cells are cultured at 37° C. with 5% CO₂ for 120 hours, the supernatant is collected and purified.

### EXAMPLE 4

This example provides a method for preparing a detection kit containing a chicken-derived recombinant full-length monoclonal IgY antibody against human TK1:

The kit includes a calibrator, a quality control, a blocking agent, a magnetic particle reagent coupled to the first antibody, a biotin-labeled second antibody, a streptavidin-labeled alkaline phosphatase, a luminescent substrate, an anti-reagent, a diluent and a washing solution. The preparation method is as follows:

### 1. Magnetic Particle Reagent Coupled to the First Antibody

1) Putting the fully mixed tosyl magnetic particle concentrate into the reaction bottle, waiting until all the magnetic particles are adsorbed by the magnetic field, removing the supernatant, adding 10 times the volume of magnetic particle activation buffer to the reaction bottle, shaking and washing for 10 min, then placing the reaction bottle in the magnetic field for 15 min, removing the supernatant; repeating the washing of the magnetic particles twice; finally diluting the magnetic particle solution to 1-30 mg/ml, mixing well and set aside;
2) Adding the first antibody to the tosyl magnetic particle solution prepared in step 1) according to a mass ratio of tosyl magnetic particle solution: first antibody (recombinant chicken anti-human TK1 full-length IgY monoclonal antibody)=100:1 to perform a ligation reaction, adding 1/10 of the total volume of magnetic particle catalytic buffer, and reacting at 37° C. for 18 hours in a mixed state;
3) Adding 10% BSA of 1/20 of the total volume of the solution to the magnetic particle solution prepared in step 2), and reacting at 37° C. for 6 hours in a mixed state;
4) Placing the reaction flask in a magnetic field for 15 minutes, cleaning the tosyl magnetic particles 3 times with magnetic particle cleaning solution after the tosyl magnetic particles are adsorbed into the magnetic field, then adjusting to 1-20 mg/ml and storing at 4° C. to prepare the first antibody coupled with magnetic particles.

The method for preparing the magnetic particle activation buffer includes: dissolving 1-10 g of boric acid in 900 ml of deionized water, adjusting pH to 8-10 with NaOH, diluting to 1 L and filtering with a 0.45 μm filter membrane.

The method for preparing the magnetic particle catalytic buffer includes: dissolving 30-300 g of ammonium sulfate in 1 L of magnetic particle activation buffer, and filtering with 0.45 μm filter membrane after complete dissolution.

The magnetic particle cleaning solution is a TBST buffer with PH 7.4.

### 2. Secondary Antibody Labeled with the Biotin

Using PBS to prepare the recombinant chicken anti-human TK1 full-length IgY monoclonal antibody into a 0.1-20 mg second antibody solution, which includes: using DMSO to prepare a 5-50 mg biotin solution, adding the biotin solution to the antibody solution and mixing well, and reacting in an ice bath for 2 hours or at room temperature for 30 minutes to prepare second antibody labeled with the biotin.

### 3. Luminescence Substrate

Alkaline phosphatase luminescent substrate No. 1, item number: APSUB-1 (Beijing Avid Biotechnology Co., Ltd.).

### 4. Blocking Agent

Skimmed milk powder is provided by Thermo Fisher Scientific (China) Co., Ltd.

### 5. Diluent

Dissolving 0.1 g-10 g of blocking agent in 1 L Tris buffer, adding 0.1-5 ml of preservative, completely dissolving and filtering with 0.22 μm filter membrane.

### 6. Biotinylated Antibody Working Solution

The biotinylated anti-thymidine kinase 1-IgY polyclonal antibody is diluted to a final concentration of 0.05-15 μg/mL.

### 7. Washing Solution

pH 7.4 TBST buffer (30 times concentrated solution)

### 8. Streptavidin-Labeled Alkaline Phosphatase

Streptavidin-labeled alkaline phosphatase was purchased from Invitrogen and diluted 50,000 times with a diluent.

### EXAMPLE 5

This example provides a method for preparing a drug containing a chicken-derived recombinant full-length monoclonal IgY antibody against human TK1:
1. Construction of a dual-expression cassette vector for recombinant chicken anti-human TK1 full-length IgY gene: optimizing amino acid sequences of the heavy chain and light chain of the 5^{#} antibody into CHO codon synthetic genes, designing Hind III and Xma I restriction sites at both ends of the heavy chain, and designing BsiWIand EcoRIrestriction sites at both ends of the light chain. The heavy chain is loaded and inserted into the pEE 6.4 vector with the designed restriction sites, and the light chain is loaded and inserted into the pEE 12.4 vector with the designed restriction sites. The two expression vectors are then digested with NotIand SalI, and the heavy chain ORF from pEE 6.4 is recovered and ligated into pEE 12.4, and linearized with PVU I.
2. CHO transfection and pressurization: CHO cells are revived and the cell state is adjusted to the best. The linearized expression vector is electroporated into the cells using the GenePulserXcell# system, and the culture medium MSX is supplemented the next day for three weeks of pressurization. After most of the cells die, new clones grow. 30 highly expressed cell wells are selected and transferred to a 24-well plate, then transferred to a 6-well plate. The cells are frozen after the cell density reaches 80%.
3. Screening of monoclonal cell lines: Multiple pressurization with small gradients is conducive to obtaining highly expressed cell lines, but the expression of cell lines is unstable. After the screening pressure is removed, the expression level often decreases, preparing for the next step of limiting dilution screening. The pressurization concentration can be flexibly changed according to the actual culture situation. It is temporarily set to pressurize for three rounds, which can be adjusted according to the actual situation.

Screening by limiting dilution method: Explore the density of cell plating: start with 1000 cells/well and dilute in multiples. Spread 10 plates at each density, culture at 37° C. with 5% CO₂, and observe. Take the supernatant of the 96-well plate for ELISA, select the clone with high titer to culture in the 12-well plate. Take the supernatant of the 12-well plate for ELISA, select the clone with high titer to culture in the 6-well plate. Take the supernatant of the 6-well plate for ELISA, select the clone with high titer to culture in the shake flask, observe under the microscope, cell density, and viability to comprehensively evaluate the cell state. 10-20 cell lines with a viability greater than 95% are frozen; take 1.5-2.0 mL of supernatant for ELISA and HPLC to evaluate antibody expression, and HPLC to evaluate the final antibody yield (standard ≥5 g/L).
4. Stability observation:

At each culture generation, when the cell viability drops below 50%, take 1.5-2.0 mL of supernatant to test ELISA and HPLC to evaluate antibody expression. HPLC is used to evaluate the final antibody yield. The final yield is required to drop by less than 10%. If it meets the requirements, a cell bank can be established.
5. Stable cell line fermentation and purification.

Cell lines with a yield of more than 5 g/L are selected for fermentation production. The cell supernatant is purified and impurities are removed to ensure that the product meets the antibody drug standards.

The present application is to apply an improved phage display method, using the C-terminal 195-225 polypeptide of human TK1 to develop a chicken anti-human TK1 full-length-IgY monoclonal antibody. Using the improved phage display method, the positive cloning rate is as high as 98%. A super large library of 3.4×10⁹ pfu/ml was quickly constructed within 3 weeks, which can completely eliminate environmental phage contamination. Therefore, we successfully obtained a single-chain antibody that meets the requirements of the inventor and obtained the variable region sequence of the antibody heavy and light chains. After analyzing the obtained antibody fragments, the full-length IgY antibody was constructed, and then the antibody was expressed through a eukaryotic expression system and purified. The antibody was verified to have high specificity and high sensitivity by detection methods such as ELISA titer, immunoblotting, and immunohistochemistry.

More importantly, serological identification pointed out that the two serum test kits we assembled were used for serum testing, and the test results showed that the two methods were in line with the correlation coefficient as high as r=0.857. In the future, the fully automatic chemiluminescence instrument will replace the previous semi-automatic enhanced chemiluminescence immunoassay system, and the new generation of TK1-IgY recombinant antibody kits will be run on the fully automatic instrument, which can meet the detection needs of large-scale early tumor risk screening. At present, TK1-IgY-rmAb has been successfully constructed using phage display library technology and run on the new fully automatic instrument, which has not been reported yet and is an international first.

In order to confirm that chicken-derived recombinant full-length monoclonal IgY antibody against human TK1 (hTK1-IgY-rmAb) can replace chicken anti-human TK1 natural full-length IgY polyclonal antibody (hTK1-IgY-pAb), the inventors used hTK1-IgY-rmAb and hTK1-IgY-pAb to detect the concentration of STK1p in 95 voluntary blood donors (self-reported to have no tumor or infectious disease) by enhanced chemiluminescence dot blot (ECL-dot blot). As shown in FIG. 5, the coincidence rate of the Pearson test was r=0.988, which confirmed that the replacement of hTK1-IgY-pAb with hTK1-IgY-rmAb was credible.

The collection and use of serum and tissue specimens in the present application were carried out in accordance with the Declaration of the 1964 Declaration of Helsinki and the Tripartite Guidelines for Clinical Practice of the International Conference on Harmonization. This study was approved by Changzhou Cancer Hospital, ethics number 2020-SY-032. All participants provided informed consent before entering the study.

Recently, the advantages of preparing chicken-derived recombinant monoclonal antibodies and immunotherapy biomolecules have only been fully recognized. Recombinant monoclonal antibodies from mammalian sources are not as specific as those from chickens; Compared with TK1-IgY polyclonal antibodies, we found that the human TK1-IgY recombinant monoclonal antibody has higher affinity and specificity in recognizing a single unique epitope of human TK1. The present application objectively expounds the advantages of the implementation plan for preparing chicken-derived human TK1-IgY recombinant monoclonal antibodies. This will achieve large-scale production of antibodies with strong specificity, high sensitivity and high stability, ensuring the large-scale detection needs in population physical examination screening and clinical tumors.

The above-described embodiments are only some embodiments of the present invention, which are used to explain the present application and are not used to limit the scope of the present application. The name of the present application has been described through specific embodiments. Those skilled in the art can refer to the content of the present application to appropriately change the raw materials and conditions and other aspects to achieve corresponding other purposes, and the relevant changes do not deviate from the content of the present application. All similar replacements and modifications are obvious to those skilled in the art and are deemed to be included in the scope of the present application.

What is claimed is:

1. A chicken-derived recombinant full-length monoclonal IgY antibody against human tymidine kinase 1 (TK1), comprising a chicken-derived recombinant IgY single-chain antibody against human TK1 comprising:
a heavy chain, comprising a first heavy chain variable region, a second heavy chain variable region and a third heavy chain variable region;
a linkage peptide; and
a light chain, comprising a first light chain variable region, a second light chain variable region and a third light chain variable region;
wherein the first heavy chain variable region comprises the amino acid sequence selected from one of SEQ ID NO: 1 to SEQ ID NO: 3, the second heavy chain variable region comprises the amino acid sequence selected from one of SEQ ID NO: 4 to SEQ ID NO: 6, and the third heavy chain variable region comprises the amino acid sequence selected from one of SEQ ID NO: 7 to SEQ ID NO: 9;
the first light chain variable region comprises the amino acid sequence selected from one of SEQ ID NO: 10 to SEQ ID NO: 14, the second light chain variable region comprises the amino acid sequence selected from one of SEQ ID NO: 15 to SEQ ID NO: 19, and the third light chain variable region comprises the amino acid sequence selected from one of SEQ ID NO: 20 to SEQ ID NO: 23.

2. The chicken-derived recombinant full-length monoclonal IgY antibody against human TK1 of claim 1, further comprising the amino acid sequence selected from one of SEQ ID NO: 24 to SEQ ID NO: 28.

3. The chicken-derived recombinant full-length monoclonal IgY antibody against human TK1 of claim 2, further comprising the amino acid sequence of SEQ ID NO: 28.

4. A recombinant vector, comprising a nucleic acid encoding the amino acid sequence of the chicken-derived recombinant full-length monoclonal IgY antibody against human TK1 of claim 1, wherein a vector for constructing the recombinant vector is a PTT5 plasmid.

5. A cell containing a recombinant vector, comprising the recombinant vector of claim 4, wherein a stable cell line is constructed using Chinese hamster ovary (CHO) cells.

6. A test card, a test strip or a test kit, comprising the chicken-derived recombinant full-length monoclonal IgY antibody against human TK1 of claim 1.

7. A drug, comprising the chicken-derived recombinant full-length monoclonal IgY antibody against human TK1 of claim 1.

8. A method for screening the chicken-derived recombinant full-length monoclonal IgY antibody against human tymidine kinase 1 (TK1) of claim 1, comprising:
isolating mRNA of the chicken-derived recombinant full-length monoclonal IgY antibody against human TK1 from hen spleen cells;
performing polymerase chain reaction (PCR) reverse transcription;
performing PCR overlap to obtain the full-length scFv gene;
splicing and concentrating scFv;
electrotransformation in a highly active bacterial competent medium;
phage antibody packaging; and
screening.

9. The method of claim 8, wherein:
the isolating mRNA of the chicken-derived recombinant full-length monoclonal IgY antibody against human TK1 from hen spleen cells comprises:
quickly taking spleen cells within 15 minutes after the hen is killed, homogenizing with TriZol solution, and isolating total RNA with chloroform and isopropanol;
the performing PCR reverse transcription comprises:
using oligodT as a primer to perform a reverse transcription reaction to prepare cDNA for subsequent amplification of antibody genes;
the performing PCR overlap to obtain the full-length scFv gene comprises:
amplifying genes in the heavy chain variable region and light chain variable region using degenerate primers, and assembling the genes into scFv genes by overlapping PCR;
the splicing and concentrating scFv comprises:
isolating the PCR products, cutting the PCR products with Sfi I and Not I, and ligating the PCR products into a phagemid vector using T4 DNA ligase;
the electrotransformation in a highly active bacterial competent medium comprises:
desalting a ligation mixture and transforming the ligation mixture via electroporation into TG1 competent cells;
the phage antibody packaging comprises:
adding helper phage to the phagemid vector for packaging, followed by precipitation and titration of the resulting phagemid vector;
the screening comprises:
adding the phage library to a screening plate containing the target TK1, washing, incubating, digesting and eluting the bound phage, amplifying and screening, taking out a single colony for inoculation and culture, adding a helper phage for culture, and performing enzyme-linked immunosorbent assay (ELISA) and DNA sequencing on the phage; and
according to results of the DNA sequencing, recombining a single-chain antibody, based on the main chain of heavy chain and light chain sequence of the IgY full-length antibody, constructing a full-length antibody sequence through continuous PCR and cloning steps, and loading the full-length antibody sequence into a eukaryotic expression vector.

10. A detection method, comprising: detecting, via enhanced chemiluminescence (ECL) dot blot method or chemiluminescence immunoassay, TK1 concentration in body fluid using the chicken-derived recombinant full-length monoclonal IgY antibody against human TK1 of claim 1.

11. The detection method of claim 10, wherein the body fluid is one selected from blood, urine, saliva, cerebrospinal fluid, and effusion.
